(19) United States
(12) Patent Application Publication
(10) Pub. No.: US 2010/0143276 A1
(43) Pub. Date: Jun. 10, 2010

(54) Ester solvents derived from 4-carboxy-2-pyrrolidinone formulated into UV-screening cosmetic compositions

(75) Inventors: Hervé Richard, Gagny (FR); Benoit Muller, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/654,032

(22) Filed: Dec. 8, 2009

Related U.S. Application Data
(60) Provisional application No. 61/193,647, filed on Dec. 12, 2008.

(30) Foreign Application Priority Data
Dec. 8, 2008 (FR) 0858337

(51) Int. Cl. A61K 8/49; A61K 8/37; A61Q 17/04

(52) U.S. Cl. 424/60; 424/59

(57) ABSTRACT

Stable UV-screening cosmetic compositions contain at least one ester solvent derived from 4-carboxy-2-pyrrolidinone, of formula (I):

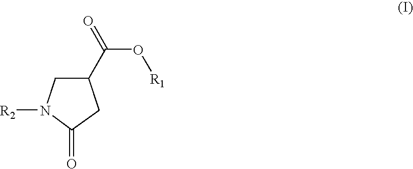

in which:
R₁ is a linear or branched C₁-C₂₀ alkyl radical, and
R₂ is a linear or branched C₁-C₂₀ alkyl radical optionally containing a C₅-C₆ ring member, the phenyl radical, the benzyl radical or the phenethyl radical, and at least one liquid fatty phase and at least one lipophilic UV-screening active agent, the at least one ester derivative constituting a solvent for the at least one active agent in the at least one liquid fatty phase and/or constituting an agent for improving the solubility of the at least one active agent in the at least one fatty phase.

### CROSS-REFERENCE TO PRIORITY/PROVISIONAL APPLICATIONS

This application claims priority under 35 U.S.C. §119 of FR 0858337, filed Dec. 8, 2008 and §120 of U.S. Provisional Application No. 61/193,647, filed Dec. 12, 2008, each hereby expressly incorporated by reference and each assigned to the assignee hereof.

### BACKGROUND OF THE INVENTION

1. Technical Field of the Invention

The present invention relates to the formulation of at least one ester derived from 4-carboxy-2-pyrrolidinone, of formula (I), the definition of which is given hereinafter, into cosmetic compositions comprising, in a cosmetically acceptable medium, at least one liquid fatty phase and at least one lipophilic active agent, as a solvent for said active agent in said liquid fatty phase and/or as an agent for improving the solubility of said active agent in the solid fatty phase.

The present invention relates in particular to UV-screening compositions comprising, formulated into a cosmetically acceptable medium, at least one liquid fatty phase containing at least one ester derived from 4-carboxy-2-pyrrolidinone, of formula (I), and at least one lipophilic active agent.

2. Description of Background and/or Related and/or Prior Art

Many cosmetic or dermatological products are provided in various galenical forms comprising a liquid fatty phase, such as dispersions, oily lotions, oil/water, water/oil or multiple emulsions, or cream gels. Certain particularly advantageous cosmetic or dermatological active agents, such as lipophilic organic screening agents, are not readily soluble in the oily phase of these formulations and have a tendency during storage to form crystals or to precipitate, in particular in emulsions. Such phenomena are undesirable from the point of view of stability of the formulation and/or with respect to consumer comfort insofar as they can destabilize the composition and/or affect the aesthetic appearance of the product and/or lead to cosmetic discomfort upon application to the skin and/or the hair, or else to the concentration of active agents being limited in these formulations, which means that products that are sufficiently effective cannot be obtained.

This is especially the case of care formulations containing low-solubility lipophilic active agents, such as active agents selected from among aminophenol derivatives, salicylic acid derivatives, 2-amino-4-alkylaminopyrimidine 3-oxide derivatives, in particular 2-amino-4-dodecylaminopyrimidine 3-oxide, DHEA (dehydroepiandrosterone), its derivatives and its chemical precursors such as 7-hydroxy or 7-keto-DHEA, or else 3β-acetoxy-7-keto-DHEA, cholesterol and its esters, plant sterols such as phytosterols and sitosterols and their esters, pentacyclic triterpenic acids, hydroxystilbenes, flavonoids, lipophilic organic UV-screening agents in anti-sun formulations, retinol and its derivatives, carotenoids such as lycopene, and also fragrances, essential oils, hormones, vitamins, in particular vitamin E, ceramides, or mixtures thereof.

In particular, anti-sun (sunscreen) compositions are often in the form of an oil-in-water or water-in-oil emulsion, of gels or of anhydrous products which contain, at various concentrations, one or more lipophilic and/or hydrophilic, insoluble and/or soluble, organic and/or inorganic screening agents capable of selectively absorbing harmful UV radiation. These screening agents and the amounts thereof are selected according to the desired protection factor. Depending on whether they are lipophilic or, conversely, hydrophilic in nature, these screening agents can be distributed, respectively, into the fatty phase or into the aqueous phase of the final composition.

Lipophilic screening agents are commonly included in anti-sun formulations. However, for a certain number of them, their photoprotective capacity when formulated is quite limited in the usual cosmetic carriers containing oils, such as oxyethylenated or oxypropylenated fatty (mono/poly)alcohols (“Cetiol HE” from Henkel or “Witconol APM” from Witco) or else fatty esters such as C₁₂-C₁₅ alkyl benzoate (“Finsolv TN” from Finetex), fatty acid triglycerides, for example Miglyol® 812 by Dynamit Nobel, or amino acid derivatives (“Eldew SL205” from Ajinomoto), the solubility of these screening agents in these oils commonly employed in formulation not being completely satisfactory. The result of this is: either the appearance over time of crystallization in the formulations, which is detrimental to the good quality, stability and effectiveness of the anti-sun products; or the fact that the concentration of screening agents in the formulations has to be limited, thereby making it impossible to obtain sufficiently effective products.

Need therefore exists to develop novel solvents for effectively dissolving lipophilic active agents and in particular lipophilic screening agents to improve their solubility in the oils and in the carriers of cosmetic or dermatological formulations containing them, without the drawbacks indicated above.

Pyrrolidone derivatives are known as penetrating agents for active agents such as oleocanthal in WO 2008/01240 or such as asprotadil alkyl esters in U.S. Pat. No. 6,673,841.

### SUMMARY OF THE INVENTION

Surprisingly, a novel family of effective solvents has now been developed constituted of esters derived from 4-carboxy-2-pyrrolidinone, of formula (I), the definition of which is given hereinafter, which make it possible to achieve the above objective. These compounds can be incorporated into many cosmetic products.

This discovery provides the basis of the present invention.

The present invention features formulation of at least one ester derived from 4-carboxy-2-pyrrolidinone, of formula (I), the definition of which is given below, into compositions comprising, in a cosmetically acceptable medium, at least one liquid fatty phase and at least one lipophilic active agent, as a solvent for said active agent in said liquid fatty phase and/or as an agent for improving the solubility of said active agent in said fatty phase.

The present invention in particular features compositions comprising, formulated into a cosmetically acceptable medium, at least one liquid fatty phase containing at least one ester derived from 4-carboxy-2-pyrrolidinone, of formula (I), and at least one lipophilic active agent.

The present invention also features formulation of at least one derivative of formula (I) into a composition comprising, in a cosmetically acceptable medium, at least one liquid fatty phase and at least one lipophilic active agent, for improving the effectiveness of said active agent and/or the cosmetic qualities and/or the stability of said composition.

The present invention also features formulation of at least one derivative of formula (I) into a composition comprising, in a cosmetically acceptable medium, at least one liquid fatty phase and at least one lipophilic organic UV screening agent, for improving the sun protection factor.

Other characteristics, aspects and advantages of the invention will become apparent from the detailed description which follows.

The term “cosmetically acceptable” means compatible with the skin and/or its appendages, which has a pleasant color, odor and feel and which does not create any unacceptable discomfort (tingling, tautness, redness), that may dissuade the consumer from using this composition.

For the purpose of the present application, the term “liquid fatty phase” means a fatty phase that is liquid at ambient temperature (25° C.) and atmospheric pressure (760 mmHg), composed of one or more fatty substances that are liquid at ambient temperature, also known as oils, which are compatible with one another.

The term “lipophilic active agent” means any cosmetic or dermatological organic active agent that can be completely dissolved in the molecular state in a liquid fatty phase, or else can be solubilized in colloidal form (for example in micellar form) in a liquid fatty phase.

The expression “low-solubility lipophilic active agent” means any cosmetic or dermatological organic active agent having a solubility in water of less than 0.5% by weight and a solubility of less than 10% by weight in most organic solvents such as liquid paraffin, fatty alcohol benzoates and fatty acid triglycerides, for example Miglyol® 812 by Dynamit Nobel. This solubility, attained at 70° C., is defined as the amount of product in solution in the solvent at equilibrium with an excess of solid in suspension after a return to ambient temperature. It can be readily evaluated in the laboratory.

### DETAILED DESCRIPTION OF BEST MODE AND SPECIFIC/PREFERRED EMBODIMENTS OF THE INVENTION

The esters derived from 4-carboxy-2-pyrrolidinone in accordance with the invention are selected from among those corresponding to general formula (I) below:

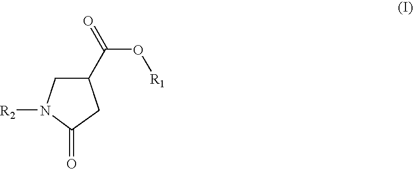

in which:

R₁ is a linear or branched C₁-C₂₀ alkyl radical, and

R₂ is a linear or branched C₁-C₂₀ alkyl radical optionally comprising a C₅-C₆ ring member, the phenyl radical, the benzyl radical or the phenethyl radical.

In formula (I), exemplary alkyl radicals include methyl, ethyl, n-propyl, isopropyl, n-butyl, isobutyl, tert-butyl, n-octyl, 2-ethylhexyl, dodecyl, hexadecyl, cyclohexyl and methylcyclohexyl radicals.

Among the compounds of formula (I), particularly exemplary are the following compounds (a) to (oo):

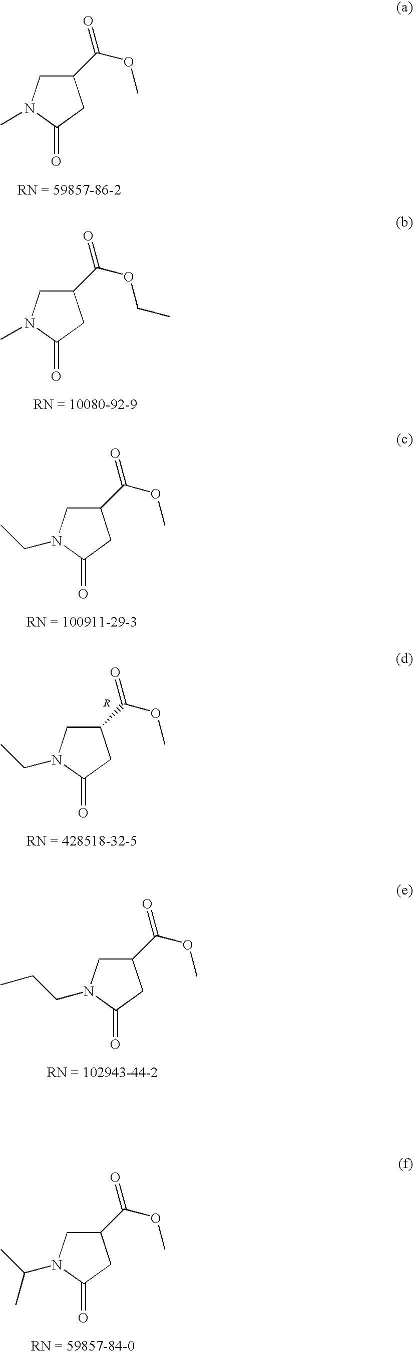

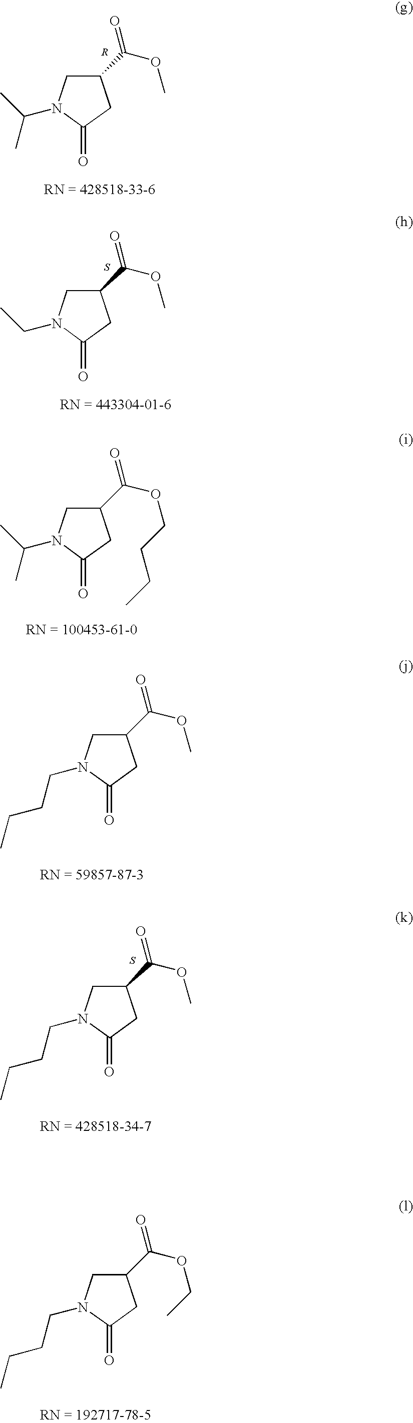

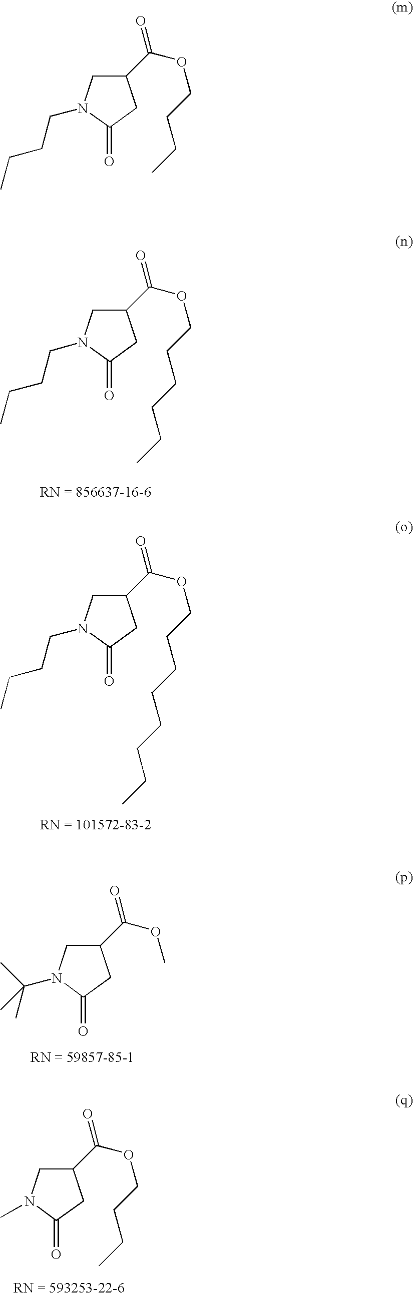

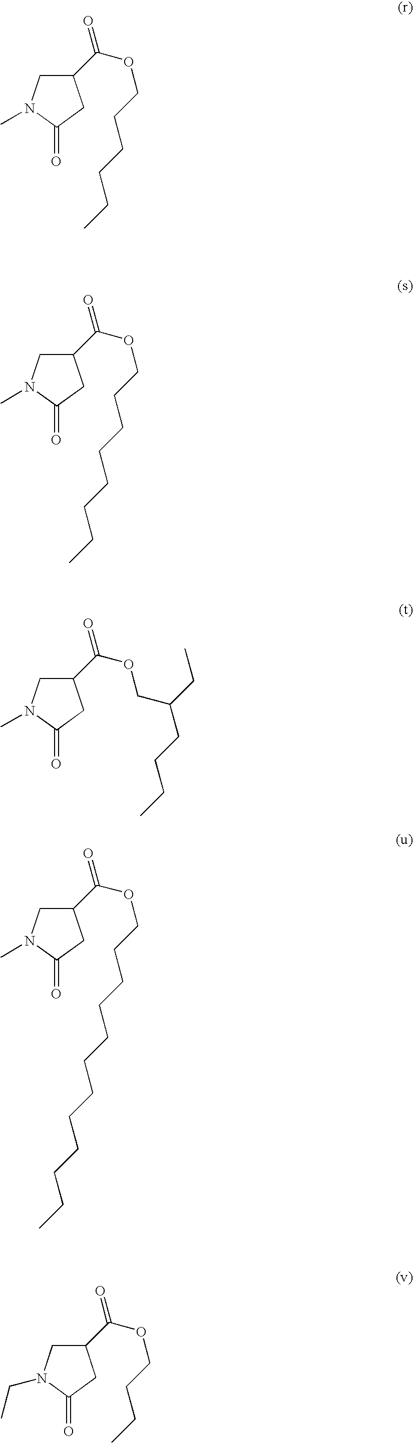

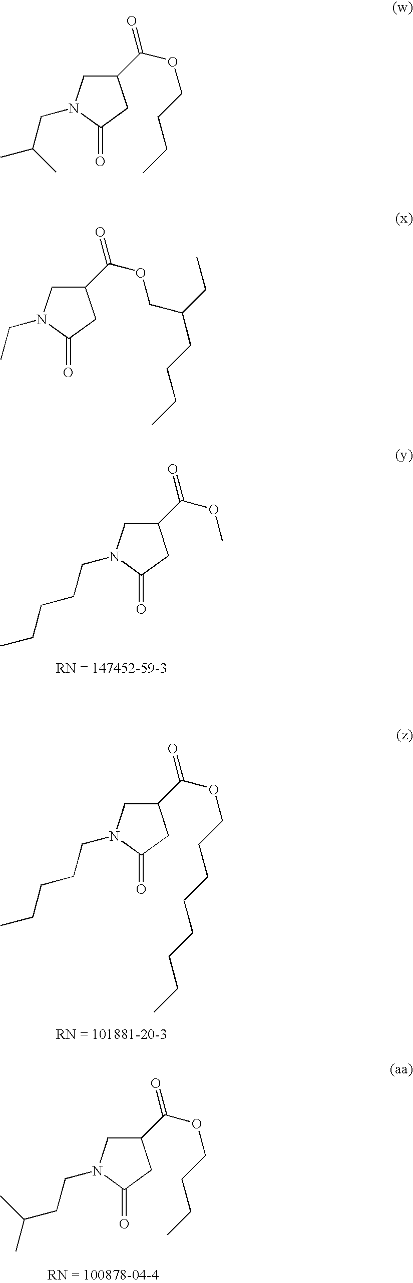

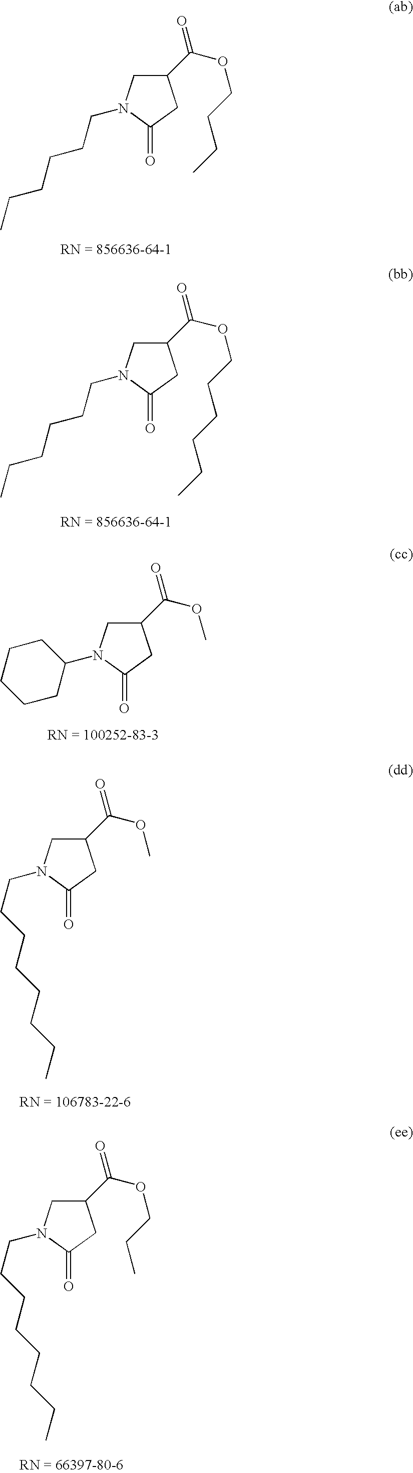

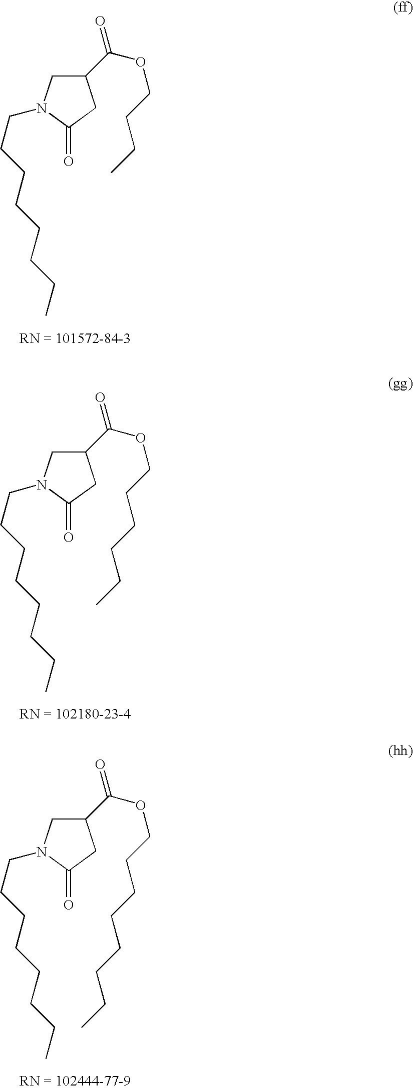

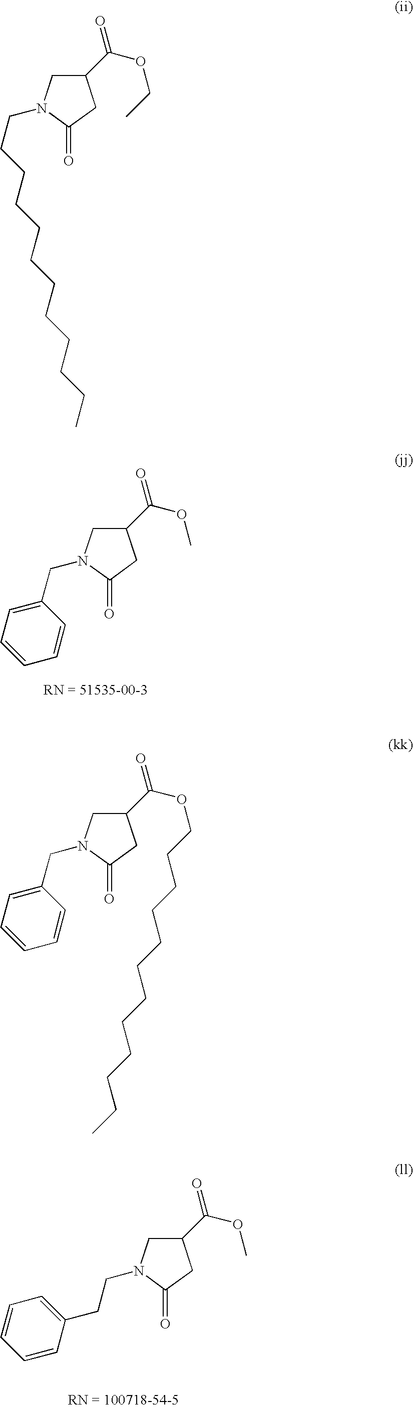

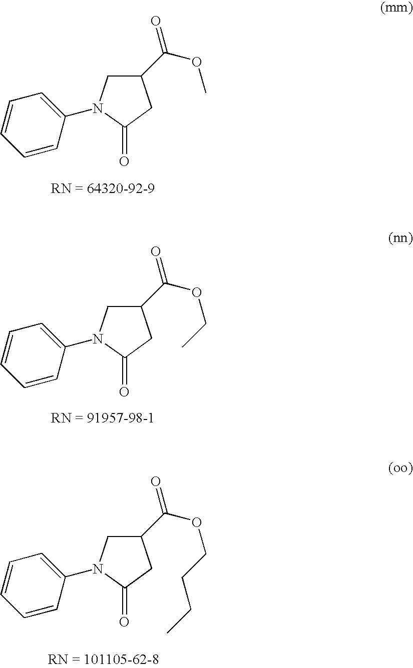

More particularly preferred are the compounds examples (t), (u), (v), (x), (j), (l), (m), (w), (n), (ab), (ii) and (kk).

The compounds of formula (I) in accordance with the invention are preferably present in the compositions of the invention at contents ranging from 1% to 30% by weight, and more preferably from 3% to 20% by weight, relative to the total weight of the composition.

The derivatives of formula (I), the syntheses of which are described in the following articles: J. Org. Chem., 26, pages 1519-24 (1961); Tetrahedron Asymmetric, 12 (23), pages 3241-9 (2001); J. Industrial & Engineering Chem., 47, pages 1572-8 (1955); J. Am. Chem. Soc., 60, pages 402-6 (1938); and in EP-0069512, U.S. Pat. Nos. 2,811,496 (1955), 2,826,588, 3,136,620, FR 2,290,199 and FR 2,696,744, are readily prepared:

either by condensation of a diester of itaconic acid of formula (II) with a primary amine of formula (III), with or without solvent at a temperature from 20° C. to 150° C. according to the following reaction scheme:

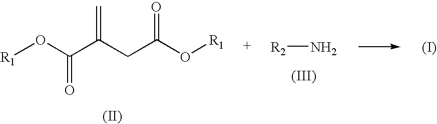

or in two stages starting from the itaconic acid of formula (IV) by condensation with the primary amine of formula (III), in the presence or absence of a solvent, such as to provide the intermediate acid of formula (V), followed by esterification of this acid of formula (V) in the presence of an excess of alcohol of formula (VI) according to the following reaction scheme:

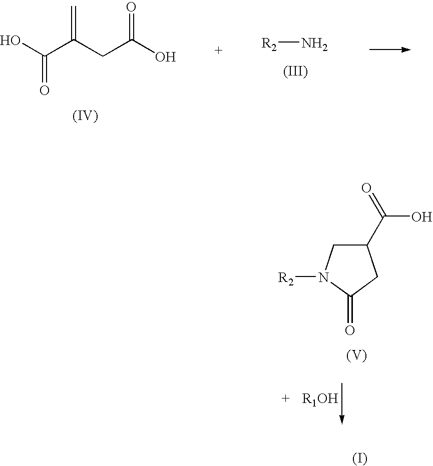

or the derivatives of formula (I) comprising an ester chain R′₁ (linear or branched C₃-C₂₀ alkyl radical) can also be obtained by transesterification of the derivatives comprising an ester chain R₁ (methyl or ethyl radical) in the presence of a linear or branched C₃-C₂₀ alcohol and of a tin catalyst or titanium catalyst according to the following reaction scheme:

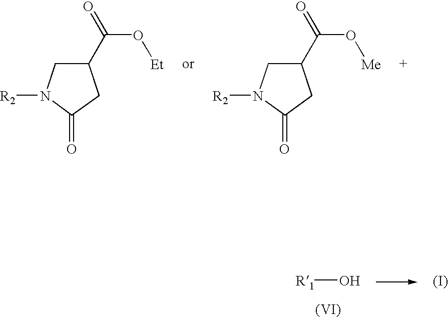

According to one particular embodiment of the invention, the derivative(s) of formula (I) is (are) present in an amount sufficient to solubilize, by itself or by themselves (without it being necessary to use another solvent), the entire amount of lipophilic active agent(s) present in the composition.

According to another particular embodiment of the invention, the derivative(s) of formula (I) constitute(s) the sole solvent for the lipophilic active agent(s).

In this case, the liquid fatty phase may be constituted of the derivative(s) of formula (I) and the lipophilic active agent(s) dissolved in said phase.

Among the lipophilic active agents in accordance with the invention, lipophilic organic screening agents will be used. These can be selected from among para-aminobenzoic acid derivatives, salicylic derivatives, cinnamic derivatives, anthranilic derivatives, dibenzoylmethane derivatives, β,β-diphenylacrylate derivatives, benzylidenecamphor derivatives, phenylbenzimidazole derivatives, benzotriazole derivatives, imidazoline derivatives, benzalmalonate derivatives, 4,4-diarylbutadiene derivatives, benzoxazole derivatives, malonitrile or malonate diphenyl butadiene derivatives, chalcones, and mixtures thereof.

Among the lipophilic UV-A screening agents capable of absorbing UV radiation from 320 to 400 nm, exemplary are:

Dibenzoylmethane Derivatives:

4-isopropyldibenzoylmethane, marketed under the trademark “Eusolex 8020” by Merck, and corresponding to the formula below:

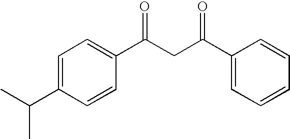

1-(4-methoxy-1-benzofuran-5-yl)-3-phenylpropane-1,3-dione, marketed by Quest under the trademark Pongamol, of formula:

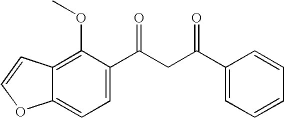

1-(4-tert-butylphenyl)-3-(2-hydroxyphenyl)propane-1,3-dione, of formula:

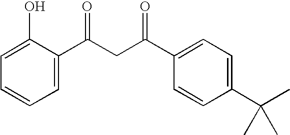

butyl methoxydibenzoylmethane marketed in particular under the trademark “Parsol 1789” by Hoffmann La Roche,

Anthranilic Derivatives:

Menthyl anthranilate, marketed under the trademark “Neo Heliopan MA” by Haarmann and Reimer,

4,4-Diarylbutadiene derivatives:

1,1-dicarboxy(2,2′-dimethylpropyl)-4,4-diphenylbutadiene.

Those preferred are among the lipophilic UV-B screening agents, capable of absorbing UV radiation from 280 to 320 nm, including:

para-Aminobenzoates:

Ethyl PABA

Ethyl dihydroxypropyl PABA

Ethylhexyl dimethyl PABA (Escalol 507 from ISP),

Salicylic Derivatives:

Homosalate, marketed under the trademark “Eusolex HMS” by Rona/EM Industries,

Ethylhexyl salicylate, marketed under the trademark “Neo Heliopan OS” by Haarmann and Reimer,

Dipropylene glycol salicylate, marketed under the trademark “Dipsal” by Scher,

TEA salicylate, marketed under the trademark “Neo Heliopan TS” by Haarmann and Reimer,

Cinnamic Derivatives:

Ethylhexyl methoxycinnamate, marketed in particular under the trademark “Parsol MCX” by Hoffmann La Roche,
Isopropyl methoxycinnamate,
Isoamyl methoxycinnamate, marketed under the trademark “Neo Heliopan E 1000” by Haarmann and Reimer,
Diisopropyl methylcinnamate,

### Cinoxate,

Glyceryl ethylhexanoate dimethoxycinnamate,
(2E)-3-(2,4,5-trimethoxyphenyl)prop-2-enoic acid having the formula below:

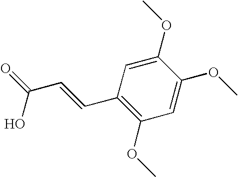

β,β′-Diphenylacrylate Derivatives:

Octocrylene, marketed in particular under the trademark “Uvinul N539” by BASF,
Etocrylene, marketed in particular under the trademark “Uvinul N35” by BASF,

Benzylidenecamphor Derivatives:

3-Benzylidenecamphor, manufactured under the trademark “Mexoryl SD” by Chimex,
Methylbenzylidenecamphor, marketed under the trademark “Eusolex 6300” by Merck,
Polyacrylamidomethyl benzylidenecamphor, manufactured under the trademark “Mexoryl SW” by Chimex,

Imidazoline Derivatives:

Ethylhexyl dimethoxybenzylidene dioxoimidazoline propionate,

Benzalmalonate Derivatives:

Polyorganosiloxanes comprising a benzalmalonate function, such as Polysilicone-15 marketed under the trademark “Parsol SLX” by Hoffmann La Roche,
Dineopentyl 4′-methoxybenzalmalonate.

Among the broad-spectrum lipophilic screening agents capable of absorbing UV-A and UV-B radiation, exemplary are:

Benzotriazole Derivatives:

Drometrizole trisiloxane, marketed under the trademark “Silatrizole” by Rhodia Chimie,
Bumetrizole, marketed under the trademark “Tinoguard AS” by Ciba-Geigy.

Diphenyl Butadiene Derivatives:

The derivatives of the class of the malonitrile or malonate diphenyl butadienes are derivatives of general formula (VII) below:

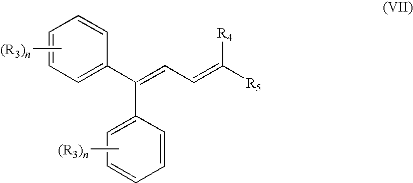

in which:

R₃ is a C₁-C₂ alkyl radical or a C₁-C₂ alkoxy radical and n is 0, 1 or 2; and

R₄ and R₅, which may be identical or different, are each —COOR₆, —(C═O)NHR₆, —(C═O)R₆ or —CN, in which R₆ is a linear or branched alkyl radical having from 1 to 12 carbon atoms, and which may contain silane, siloxane or polysiloxane groups.

Among the malonitrile or malonate diphenyl butadiene derivatives, exemplary are:
dimethyl 2-(3,3-diphenylprop-2-enylidene)malonate,
diisobutyl 2-(3,3-diphenylprop-2-enylidene)malonate,
bis(1,3-dimethylbutyl) 2-(3,3-diphenylprop-2-enylidene) malonate,
dineopentyl 2-(3,3-diphenylprop-2-enylidene)malonate,
methyl (2Z)-2-cyano-5,5-diphenylpenta-2,4-dienoate,
ethyl (trimethylsilyl)methyl (2Z)-2-(3,3-diphenylprop-2-enylidene)malonate,
(2E)-2-cyano-5,5-diphenyl-N-(3-{1,3,3,3-tetramethyl-1-[(trimethylsilyl)oxy]disiloxanyl}propyl)penta-2,4-dienamide,
ethyl 2-methyl-3-{1,3,3,3-tetramethyl-1-[(trimethylsilyl)oxy]disiloxanyl}propyl (2E)-2-(3,3-diphenylprop-2-enylidene)malonate, and
ethyl (2Z)-5,5-diphenyl-2-{[(3-{1,3,3,3-tetramethyl-1-[(trimethylsilyl)oxy]disiloxanyl}propyl)amino]carbonyl}penta-2,4-dienoate.

Among the diphenyl butadiene derivatives indicated above, particularly useful is dineopentyl 2-(3,3-diphenylprop-2-enylidene)malonate corresponding to the formula below:

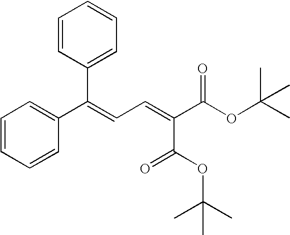

It is known practice to formulate these diphenyl butadiene derivatives into anti-sun (sunscreen) compositions; EP 0916335 describes carbon derivatives and also methods for obtaining them, and EP 1535947 and EP 1535925 describe siloxane and silane derivatives, respectively.

Chalcones:

The lipophilic screening agents of the chalcone class correspond to general formula (VIII) below:

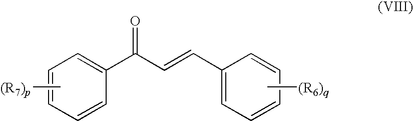

in which the R₆ and R₇ radicals denote, independently of one another, a hydrogen atom, the hydroxyl radical, a linear or branched C₁-C₁₂ alkyl or alkenyl radical, a linear or branched C₁-C₁₂ alkoxy radical or a linear or branched C₂-C₂₀ acyloxy radical; and

p and g are each 1 to 4.

Among the chalcone derivatives, particularly exemplary are:
2′-hydroxychalcone
4′-hydroxychalcone
4′-methoxychalcone
2′-hydroxy-4-methoxychalcone
2′-hydroxy-4-hexyloxychalcone
2′-hydroxy-4-methylchalcone
2′-hydroxy-3-hexyloxychalcone
2′-hydroxy-4′-hexyloxy-4-methylchalcone
2′-hydroxy-4′-hexanoyloxy-4-methoxychalcone
2′,4′,4-trihydroxy-3,3′-diallylchalcone (known as Kazonol)
2′,4′,4-trihydroxy-5′-(3-methylbut-2-ene)chalcone (known as Broussochalcone B)
2′,3′,4′,6′,4-pentahydroxychalcone (known as Carthamin).

Among the chalcone derivatives indicated above, particularly exemplary are 4′-hydroxychalcone corresponding to formula (VIIIa) below:

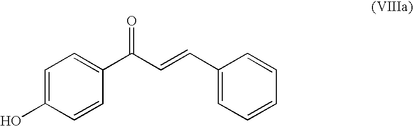

or else 2′,3′,4′,6′,4-pentahydroxychalcone (known as Carthamin) corresponding to formula (VIIIb) below:

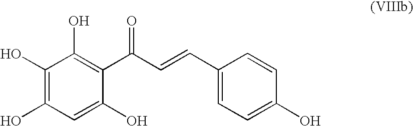

It is known to formulate these chalcone compounds in anti-sun compositions, in particular in FR 2555167, FR 2602228 and FR 2608150.

The lipophilic screening agents are generally present in the compositions according to the invention in proportions ranging from 0.01% to 20% by weight, relative to the total weight of the composition, and preferably ranging from 0.1% to 10% by weight, relative to the total weight of the composition.

Among the screening agents of which the lipophilicity is difficult, exemplary are dibenzoylmethane derivatives, benzotriazole derivatives, chalcones, and derivatives of the class of malonitrile or malonate diphenyl butadienes.

The term “low-solubility lipophilic screening agent” means any cosmetic or dermatological organic active agent which has a solubility in water of less than 0.5% by weight and a solubility of less than 10% by weight in most organic solvents such as liquid paraffin, fatty alcohol benzoates and fatty acid triglycerides, for example Miglyol® 812 by Dynamit Nobel. This solubility, attained at 70° C., is defined as the amount of product in solution in the solvent at equilibrium with an excess of solid in suspension after a return to ambient temperature. It can be readily evaluated in the laboratory.

The low-solubility lipophilic active agents in accordance with the invention may also be selected from among aminophenol derivatives, salicylic acid derivatives, N,N′-di(arylmethylene)ethylenediaminetriacetate derivatives, 2-amino-4-alkylaminopyrimidine 3-oxide derivatives, flavonoids, retinol and its derivatives, carotenoids such as lycopene, and also fragrances, essential oils, hormones, vitamins, in particular vitamin E, ceramides, or mixtures thereof.

The aminophenol derivatives employed are more particularly the derivatives of formula (IX) below:

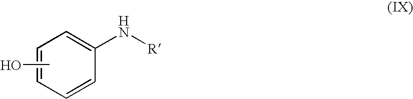

in which R′ is a radical selected from among the following radicals (a), (b) and (c):

(a) —CO—NR¹R²

(b) —CO—O—R³

(c) —SO₂R³

wherein R¹ is a hydrogen atom or an optionally hydroxylated, saturated or unsaturated, linear or branched C₁ to C₆ alkyl radical,

R² is a hydrogen atom or a radical selected from among saturated or unsaturated, linear, cyclic or branched C₁₂ to C₃₀ alkyl radicals, optionally hydroxylated, and

R³ is a radical selected from among saturated or unsaturated, linear, cyclic or branched, including fused polycyclic, C₁₂ to C₃₀ alkyl radicals, which are optionally hydroxylated.

In formula (IX), among the linear or branched R² or R³ alkyl radicals having from 1 to 30 carbon atoms, particularly exemplary are methyl, ethyl, propyl, isopropyl, butyl, tert-butyl, hexyl, octyl, nonyl, 2-ethylhexyl, dodecyl, hexadecyl, behenyl, octadecyl and 2-butyloctyl radicals. Preferably, these radicals contain from 1 to 12 carbon atoms. Even more preferably, the alkyl radical generally contains from 1 to 6 carbon atoms. As a lower alkyl radical, exemplary are methyl, ethyl, propyl, isopropyl, tert-butyl and hexyl radicals.

When it is unsaturated, a radical having one or more ethylenic unsaturations, such as more particularly the allyl radical, is preferred.

When the alkyl radical is cyclic, particularly exemplary is the cyclohexyl, cholesteryl or tert-butylcyclohexyl radical.

When it is hydroxylated, the radical preferably contains from 1 to 6 carbon atoms and from 1 to 5 hydroxyl groups. Among the monohydroxyalkyl radicals, a radical preferably containing 1 or 3 carbon atoms, in particular the hydroxymethyl, 2-hydroxyethyl, 2-hydroxypropyl or 3-hydroxypropyl radical, is preferred.

Among polyhydroxyalkyl radicals, a radical having from 3 to 6 carbon atoms and from 2 to 5 hydroxyl groups, such as 2,3-dihydroxypropyl, 2,3,4-trihydroxybutyl, 2,3,4,5-tetrahydroxypentyl and 2,3,4,5,6-pentahydroxyhexyl radicals, is preferred.

The alkoxylated radicals are alkyl radicals, as in particular described above, preceded by an oxygen atom.

Preferably, the aminophenol compounds of this invention are those for which at least one and preferably all of the conditions below are met:

the —OH function on the phenyl radical is in the ortho-position or advantageously in the para-position,

R′ is selected from among a radical of formula (a) or (b).

Among the linear or branched alkyl radicals R¹, exemplary are methyl, ethyl, propyl, isopropyl, tert-butyl and hexyl radicals.

The aminophenol compound preferably formulated in said composition is a para-aminophenol derivative; even more preferably, it is N-ethoxycarbonyl-4-para-aminophenol of formula (IXa):

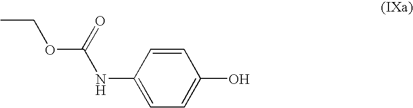

or else N-cholesteryloxycarbonyl-4-para-aminophenol of formula (IXb):

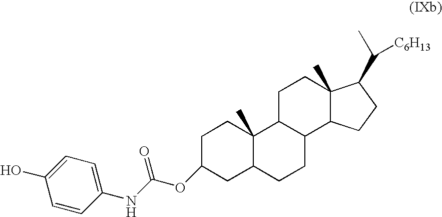

These aminophenol derivatives, and also the process for the preparation thereof, are described in WO 99/10318 and WO 99/32077.

These compounds have a more or less long hydrocarbon-based chain, preferably alkoxycarbonyl chain, attached to the nitrogen atom. They have the drawback of being poorly soluble, or even not at all soluble, in water or in the fatty phase of the type of the present invention. Their introduction into cosmetic compositions requires, as regards the compounds with a short hydrocarbon-based chain, them to be solubilized in an aqueous-alcoholic solution, which is not always desirable when the composition is intended, for example, to be applied around the eyes.

Concerning the compounds with a long hydrocarbon-based chain, these are insoluble in oils, owing to their steric bulk, and have a tendency to recrystallize from water.

The compositions according to the present invention comprising such an aminophenol compound can be administered as a depigmenting or bleaching agent in a cosmetic and/or dermatological composition.

The salicylic acid derivatives are the compounds of formula (X) below:

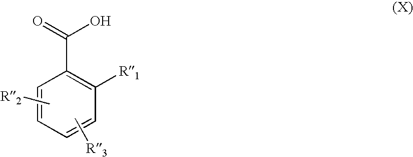

in which:

R″₁ is a hydroxyl radical or an ester of formula —O—CO—R″₄

in which R″₄ is a saturated or unsaturated, aliphatic radical having from 1 to 26 carbon atoms, preferably from 1 to 18 carbon atoms, or an amine or thiol function optionally substituted with an alkyl radical having from 1 to 18 carbon atoms, and preferably from 1 to 12 carbon atoms,

R″₂ and R″₃, independently of one another, are in the 3-, 4-, 5 or 6-position on the benzene ring and represent, independently of one another, a hydrogen atom or a radical: —(O)ₙ—(CO)ₘ—R″₅

in which n and m, independently of one another, are each an integer equal to 0 or 1, provided that R″₂ and R″₃ are not simultaneously hydrogen atoms, and R″₅ is a hydrogen, a linear, branched or cyclized, saturated aliphatic radical having from 1 to 18 carbon atoms, or an unsaturated radical having from 3 to 18 carbon atoms, bearing one to nine conjugated or unconjugated double bonds, with the proviso that the radicals may be substituted with at least one substituent selected from among halogen atoms (fluorine, chlorine, bromine, iodine), the following radicals: trifluoromethyl, hydroxyl in free form or esterified with an acid having from 1 to 6 carbon atoms, or carboxyl in free form or esterified with a lower alcohol having from 1 to 6 carbon atoms, or an aromatic radical having from 6 to 10 carbon atoms.

Preferably, the salicylic acid derivative is such that R″₅ is a saturated aliphatic radical having from 3 to 15 carbon atoms.

Preferably, the salicylic acid derivative is such that R″₅ is a hydroxyl radical.

Preferably, the salicylic acid derivative is such that R″₅ is in the 5-position on the benzene ring and R″₂ is a hydrogen atom.

According to one preferred embodiment of the invention, the salicylic acid derivatives are derivatives of 5-n-octanoylsalicylic acid, 5-n-decanoylsalicylic acid, 5-n-dodecanoylsalicylic acid, 5-n-octylsalicylic acid, 5-n-heptyloxysalicylic acid, 4-n-heptyloxysalicylic acid, 5-tert-octylsalicylic acid, 3-tert-butyl-5-methylsalicylic acid, 3-tert-butyl-6-methylsalicylic acid, 3,5-diisopropylsalicylic acid, 5-butoxysalicylic acid, 5-octyloxysalicylic acid, 5-propanoylsalicylic acid, 5-n-hexadecanoylsalicylic acid, 5-n-oleoylsalicylic acid, 5-benzoylsalicylic acid, monovalent and divalent salts thereof, and mixtures thereof.

Most particularly preferred is use of the 2-hydroxy-5-octanoylbenzoic acid marketed under the trademark “Mexoryl SAB” by Chimex; it corresponds to formula (Xa) below:

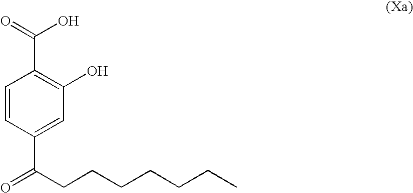

It is known practice to formulate salicylic acid compounds in topical compositions, for example, as a keratolytic agent for treating acne or as an anti-aging agent; FR-A-2,581,542 and EP-A-378,936 describe such derivatives.

The salicylic acid derivatives are highly advantageous in particular for preventing or repairing the principal manifestations of skin aging, namely fine lines and wrinkles, disruption of the “grain” of the skin, modification of the complexion of the skin and loss of firmness and of tonicity of the skin. However, the use of these derivatives poses a problem insofar as, when they are introduced without modification into topical compositions, they do not solubilize and remain in the crystalline state, rendering the application of the composition containing them ineffective for the treatment of the skin.

Generally, these derivatives are solubilized in lower alcohols, such as ethanol or isopropanol, or solvents such as octyldodecanol, certain glycols, or short-chain (less than C₁₂) fatty alcohols. However, these lower alcohols have the drawback of drying out and irritating the skin; it is therefore preferable to avoid using them in body and/or facial care products. In addition, these solubilizing agents can only be introduced in small amounts otherwise they may impair the cosmetic qualities (drying out of the skin) and the stability of the compositions containing them.

The concentration of salicylic acid derivatives of the composition according to the present invention is preferably from 0.001% to 15% by weight, more preferably from 0.1% to 5% by weight, relative to the total weight of the composition. The amount of amino acid esters will depend on the amount of salicylic acid derivatives to be solubilized. It may be from 0.01% to 90% by weight, and preferably from 0.1% to 60% by weight, relative to the total weight of the composition.

The compositions according to the invention comprising at least one salicylic derivative can be used as a cosmetic or dermatological composition, and in particular for caring for, protecting, cleansing and/or making up keratin materials of human beings (skin, lips, keratin fibers such as the hair and eyelashes), and in particular for combating the signs of skin aging and/or for smoothing out facial and/or body skin and/or for treating wrinkles and fine lines of the skin and/or for stimulating the process of epidermal renewal and/or for depigmenting and bleaching the skin and/or for treating acne and/or for treating skin disorders.

These derivatives and also the process for the preparation thereof, are described in WO 94/11338.

The derivatives of the 2-amino-4-alkylaminopyrimidine 3-oxide family are the derivatives of general formula (XI) below:

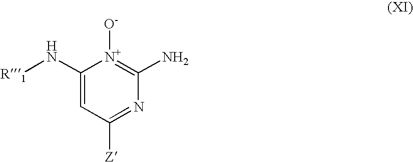

in which R′″₁ is an alkyl radical having from 1 to 20 carbon atoms, and Z′ is a hydrogen atom or an —OR′″₂ radical in which R′″₂ is an alkyl radical having from 1 to 12 carbon atoms, and also its acylated forms or its addition salts with acids.

Preferably, R′″₁ is selected from among hexyl, heptyl, octyl, nonyl, decyl, undecyl and dodecyl radicals.

Preferably, R′″₂ is selected from among ethyl, propyl, butyl, pentyl and hexyl radicals.

Even more preferably, the compound is N-(2-amino-3-oxidopyrimidin-4-yl)-N′-dodecylurea of formula (XIa) below:

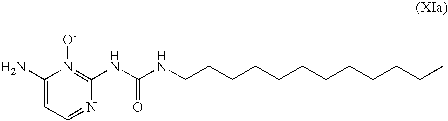

The derivatives of the 2-amino-4-alkylaminopyrimidine 3-oxide family may in particular be used in or for the preparation of a cosmetic or dermatological composition in accordance with the present invention for preventing and treating problems associated with sensitive skin and skin disturbances such as skin discomfort, tautness of the skin, skin itching, skin swelling, redness of the skin and heat sensation of the skin.

Another family of molecules which fall under the definition of molecules with low water-solubility is DHEA, derivatives thereof and chemical or metabolic precursors thereof.

DHEA, or dehydroepiandrosterone, also known as 3-beta-hydroxyandrost-5-en-17-one, or dehydroisoandrosterone, but also trans-dehydroandrosterone or prasterone, has the formula:

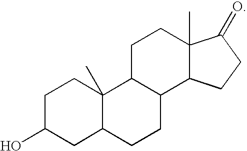

The expression “DHEA precursors to which the invention relates” means its biological precursors which are capable of being converted to DHEA during metabolism, and also its chemical precursors which can be converted to DHEA by exogenous chemical reaction.

Examples of biological precursors are Δ5-pregnenolone, 17α-hydroxypregnenolone and 17α-hydroxypregnenolone sulfate, without this list being limiting.

The expression “chemical precursors of DHEA” means in particular saponins and their derivatives such as hecogenin ((3beta,5alpha,23r)-3-hydroxyspirostan-12-one) and hecogenin acetate, diosgenin (5-spirosten-3-beta-ol), smilagenin and sarsapogenin, and also natural extracts containing them, in particular fenugreek and extracts of Dioscorea plants such as wild yam root, without this list being limiting.

The term “DHEA derivatives” means both its metabolic derivatives and its chemical derivatives. As metabolic derivatives, exemplary are Δ5-androstene-3,17-diol, and in particular 5-androstene-3β,17β-diol, Δ4-androstene-3,17-dione, 7-hydroxy-DHEA (7α-hydroxy-DHEA or 7β-hydroxy-DHEA) and 7-keto-DHEA, which is itself a metabolite of 7β-hydroxy-DHEA.

7α-Hydroxy-DHEA is, with 5-androstene-3β,17β-diol, a major metabolite of DHEA, obtained by the action of 7α-hydroxylase on DHEA. 7β-Hydroxy-DHEA is a minor metabolite of DHEA, obtained by the action of 7β-hydroxylase on DHEA.

The 7-hydroxy-DHEA preferably formulated into the compositions according to the present invention is 7α-hydroxy-DHEA. A process for preparing this compound is described in FR 2 771 105 and WO 94/08588.

As chemical derivatives of DHEA, exemplary are DHEA salts, and in particular water-soluble salts such as DHEA sulfate; DHEA esters such as esters of hydrocarboxylic acids and of DHEA, in particular those described in U.S. Pat. No. 5,736,537, or else DHEA salicylate, DHEA acetate, DHEA valerate (or n-heptanoate) and DHEA enanthate.

Also exemplary are DHEA carbamates, 2-hydroxymalonate esters of DHEA and amino acid esters of DHEA. Finally, also exemplary are 3β-acetoxy-7-oxo-DHEA which can in particular be prepared as described in U.S. Pat. Nos. 5,869,709 and 6,111,118. This list is obviously not limiting.

The concentration of DHEA-based compound in the compositions according to the present invention can advantageously range from 0.001% to 30% by weight, preferably from 0.01% to 20%, and even more preferably from 0.01% to 10% by weight, relative to the total weight of the composition. These compounds will be in solubilized form from 20° C. and 90° C.

According to a specific embodiment of the invention, the derivative(s) of formula (I) in accordance with the invention is (are) used as the sole solvent for said lipophilic active agent(s).

The compositions in accordance with the invention may also comprise other organic UV screening agents that are active in the UV-A range and/or the UV-B range and that are water-soluble or else insoluble in the cosmetic solvents commonly used.

Among the water-soluble UV-A screening agents capable of absorbing UV radiation from 320 to 400 nm, exemplary are:

terephthalylidene dicamphor sulfonic acid, marketed under the trademark “Mexoryl SX” by Chimex,

bisbenzoazolyl derivatives as described in EP-669,323 and U.S. Pat. No. 2,463,264, and more particularly the compound disodium phenyl dibenzimidazole tetrasulfonate, marketed under the trademark “Neo Heliopan AP” by Haarmann and Reimer.

Among the water-soluble UV-B screening agents capable of absorbing UV radiation from 280 to 320 nm, exemplary are p-aminobenzoic acid (PABA) derivatives such as:

PABA,

Glyceryl PABA, and

PEG-25 PABA, marketed under the trademark “Uvinul P25” by BASF,

Phenylbenzimidazole sulfonic acid, marketed in particular under the trademark “Eusolex 232” by Merck,

Ferulic acid,

Salicylic acid,

DEA methoxycinnamate,

Benzylidenecamphorsulfonic acid, marketed under the trademark “Mexoryl SL” by Chimex,

Camphor benzalkonium methosulfate, marketed under the trademark “Mexoryl SO” by Chimex, and

Among the water-soluble UV-A and UV-B screening agents, exemplary are:

Benzophenone-4, marketed under the trademark “Uvinul MS40” by BASF,

Benzophenone-5, and

Benzophenone-9.

Among the insoluble screening agents, exemplary are:

methylene bisbenzotriazolyl tetramethylbutylphenol, marketed in solid form under the trademark “Mixxim BB/100” by Fairmount Chemical or in micronized form in aqueous dispersion under the trademark “Tinosorb M” by Ciba Specialty Chemicals,

the symmetrical triazine screening agents described in U.S. Pat. No. 6,225,467, WO 2004/085412 (see compounds 6 and 9) or the document “Symmetrical Triazine Derivatives”, IP.COM Journal, IP.COM INC., West Henrietta, N.Y., US (20 Sep. 2004), more particularly the 2,4,6-tris(biphenyl)-1,3,5-triazines (especially 2,4,6-tris(biphenyl-4-yl)-1,3,5-triazine) and 2,4,6-tris(terphenyl)-1,3,5-triazine, which is included in WO 06/035000, WO 06/034982, WO 06/034991, WO 06/035007, WO 2006/034992, WO 2006/034985.

Of course, those skilled in the art will take care to select the optional additional screening agent(s) and/or the amounts thereof in such a way that the advantageous properties intrinsically associated with the compositions in accordance with the invention are not, or are not substantially, impaired by the addition(s) envisaged.

The compositions according to the invention may also contain artificial tanning and/or browning agents for the skin (self-tanning agents) and more particularly dihydroxyacetone (DHA). These are preferably present in amounts ranging from 0.1% to 10% by weight relative to the total weight of the composition.

The aqueous compositions conforming to the present invention may moreover comprise conventional cosmetic adjuvants in particular selected from among fatty substances, organic solvents, ionic or non-ionic, hydrophilic or lipophilic thickeners, demulcents, humectants, opacifiers, stabilizers, emollients, silicones, anti-foaming agents, fragrances, preservatives, anionic, cationic, non-ionic, zwitterionic or amphoteric surfactants, active agents, fillers, polymers, propellants and basifying or acidifying agents, or any other ingredient commonly used in the cosmetics and/or dermatological field.

The fatty substances may be composed of an oil or a wax other than the apolar waxes as defined previously, or mixtures thereof. The term “oil” means a compound which is liquid at ambient temperature. The term “wax” means a compound which is solid or substantially solid at ambient temperature, and of which the melting point is generally greater than 35° C.

As oils, exemplary are mineral oils (paraffin); vegetable oils (sweet almond oil, macadamia oil, blackcurrant seed oil, jojoba oil); synthetic oils such as perhydrosqualene, alcohols, fatty amides (such as isopropyl lauroyl sarcosinate marketed under the trademark “Eldew SL-205” by Ajinomoto), fatty acids or esters such as the benzoate of C₁₂-C₁₅ alcohols marketed under the trademark “Finsolv TN” or “Witconol TN” by Witco, 2-ethylphenyl benzoate such as the commercial product marketed under the trademark “X-Tend 226®” by ISP, octyl palmitate, isopropyl lanolate, triglycerides, including those of capric/caprylic acids, dicaprylyl carbonate marketed under the trademark “Cetiol CC” by Cognis, oxyethylenated or oxypropylenated fatty esters and ethers; silicone oils (cyclomethicone, polydimethylsiloxanes or PDMSs) or fluoro oils, and polyalkylenes.

As waxy compounds, exemplary are carnauba wax, beeswax, hydrogenated castor oil, polyethylene waxes and polymethylene waxes such as that marketed under the trademark “Cirebelle 303” by Sasol.

Among the organic solvents, exemplary are lower polyols and alcohols. The lower polyols may be selected from among glycols and glycol ethers such as ethylene glycol, propylene glycol, butylene glycol, dipropylene glycol and diethylene glycol.

As hydrophilic thickeners, exemplary are carboxyvinyl polymers such as carbopols (carbomers) and pemulens (acrylate/C₁₀-C₃₀ alkyl acrylate copolymers); polyacrylamides such as for example the crosslinked copolymers marketed under the trademarks “Sepigel 305” (CTFA name: polyacrylamide/C₁₃₋₁₄ isoparaffin/Laureth 7) or “Simulgel 600” (CTFA name: acrylamide/sodium acryloyldimethyltaurate copolymer/isohexadecane/polysorbate 80) by Seppic; polymers and copolymers of 2-acrylamido-2-methylpropanesulfonic acid optionally crosslinked and/or neutralized, such as poly(2-acrylamido-2-methylpropanesulfonic acid) by Hoechst under the trademark “Hostacerin AMPS” (CTFA name: ammonium polyacryloyldimethyl taurate) or “Simulgel 800” by Seppic (CTFA name: sodium polyacryloyldimethyl taurate/polysorbate 80/sorbitan oleate); copolymers of 2-acrylamido-2-methylpropanesulfonic acid and hydroxyethyl acrylate such as “Simulgel NS” and “Sepinov EMT 10” by Seppic; cellulose derivatives such as hydroxyethyl cellulose; polysaccharides and especially gums such as xanthan gum; and mixtures thereof.

As lipophilic thickeners, exemplary are synthetic polymers such as poly(C₁₀-C₃₀ alkyl acrylates) marketed under the trademark “Intelimer IPA 13-1” and “Intelimer IPA 13-6” by Landec or else modified clays such as hectorite and its derivatives, such as the products marketed under the trademark “Bentone”.

Among the active agents, exemplary are:

vitamins (C, K, PP, etc.) and derivatives or precursors thereof, alone or as mixtures;

agents for combating pollution and/or free-radical scavengers; depigmenting agents and/or propigmenting agents;

anti-glycation agents;

calmatives;

NO-synthase inhibitors;

agents for stimulating the synthesis of dermal or epidermal macromolecules and/or for preventing their degradation;

agents for stimulating fibroblast proliferation;

agents for stimulating keratinocyte proliferation;

myorelaxants;

tensioning agents;

matting agents;

keratolytic agents;

desquamating agents;

moisturizers;

anti-inflammatory agents;

agents that act on the energy metabolism of cells;

insect repellents;

substance P antagonists or CRGP antagonists;

agents for preventing hair loss and/or for hair regrowth;

anti-wrinkle agents.

Of course, those skilled in the art will take care to select the optional additional compound(s) mentioned above and/or the amounts thereof in such a way that the advantageous properties intrinsically associated with the compositions in accordance with the invention are not, or are not substantially, impaired by the addition(s) envisaged.

The compositions according to the invention may be formulated according to the techniques well known to one skilled in the art. They may be in particular in the form of a simple or complex (O/W, W/O, O/W/O or W/O/W) emulsion such as a cream, a milk or a cream-gel; in the form of an aqueous gel; or in the form of a lotion. They may also be packaged in an aerosol and be in the form of a foam or spray.

Preferably, the compositions according to the invention are in the form of an oil-in-water or water-in-oil emulsion.

The emulsions generally contain at least one emulsifier selected from among amphoteric, anionic, cationic or non-ionic emulsifiers, used alone or as a mixture. The emulsifiers are selected in an appropriate manner according to the emulsion to be obtained (W/O or O/W). The emulsions may also contain other types of stabilizers such as for example fillers, gelling polymers or thickeners.

As emulsifying surfactants that can be used for preparing W/O emulsions, exemplary are alkyl esters or ethers of sorbitan, of glycerol or of sugars; silicone surfactants, for example dimethicone copolyols, such as the mixture of cyclomethicone and of dimethicone copolyol marketed under the trademark “DC 5225 C” by Dow Corning, and alkyldimethicone copolyols such as laurylmethicone copolyol marketed under the trademark “Dow Corning 5200 Formulation Aid” by Dow Corning; cetyl dimethicone copolyol such as the product marketed under the trademark “Abil EM 90R” by Goldschmidt and the mixture of cetyl dimethicone copolyol, polyglycerol isostearate (4 mol) and hexyl laurate marketed under the trademark “Abil WE O9” by Goldschmidt. It is possible to also add thereto one or more co-emulsifiers, which advantageously may be selected from among the group comprising alkylated polyol esters.

As alkylated polyol esters, especially exemplary are polyethylene glycol esters, for example PEG-30 dipolyhydroxystearate such as the product marketed under the trademark “Arlacel P135” by ICI.

As glycerol and/or sorbitan esters, exemplary are polyglycerol isostearate, such as the product marketed under the trademark “Isolan GI 34” by Goldschmidt; sorbitan isostearate, such as the product marketed under the trademark “Arlacel 987” by ICI; sorbitan isostearate and glycerol such as the product marketed under the trademark “Arlacel 986” by ICI, and mixtures thereof.

For the O/W emulsions, exemplary emulsifiers are non-ionic emulsifiers such as oxyalkylenated (more particularly polyoxyethylenated) fatty acid and glycerol esters; oxyalkylenated fatty acid and sorbitan esters; oxyalkylenated (oxyethylenated and/or oxypropylenated) fatty acid esters such as the PEG-100 stearate/glyceryl stearate mixture marketed, for example, by ICI under the trademark “Arlacel 165”; oxyalkylenated (oxyethylenated and/or oxypropylenated) fatty alcohol ethers; sugar esters such as sucrose stearate; fatty alcohol and sugar ethers, especially alkylpolyglucosides (APGs) such as decyl glucoside and lauryl glucoside marketed, for example, by Henkel under the respective names “Plantaren 2000” and “Plantaren 1200”, cetostearyl glucoside, optionally as a mixture with cetostearyl alcohol; marketed for example under the trademark “Montanov 68” by Seppic, under the trademark “Tegocare CG90” by Goldschmidt and under the trademark “Emulgade KE3302” by Henkel, and also arachidyl glucoside, for example in the form of the mixture of arachidyl and behenyl alcohols and arachidyl glucoside marketed under the trademark “Montanov 202” by Seppic. According to one particular embodiment of the invention, the mixture of alkyl polyglucoside as defined above with the corresponding fatty alcohol may be in the form of a self-emulsifying composition, as described, for example, in WO-A-92/06778.

Among the other emulsion stabilizers, more particularly exemplary are polymers of isophthalic acid or sulfoisophthalic acid, and in particular phthalate/sulfoisophthalate/glycol copolymers, for example, the diethylene glycol/phthalate/isophthalate/1,4-cyclohexanedimethanol copolymer (INCI name: Polyester-5) marketed under the trademarks “Eastman AQ polymer” (AQ35S, AQ38S, AQ55S, AQ48 Ultra) by Eastman Chemical.

When an emulsion is involved, the aqueous phase of the latter may comprise a non-ionic vesicular dispersion prepared according to known processes (Bangham, Standish and Watkins, J. Mol. Biol., 13, 238 (1965), FR-2,315,991 and FR-2,416,008).

The compositions according to the invention find applications in a large number of treatments, in particular cosmetic treatments, for the skin, the lips and the hair, including the scalp, in particular for protecting and/or caring for the skin, the lips and/or the hair, and/or for making up the skin and/or the lips.

The present invention thus features application of the subject compositions, for the cosmetic treatment of the skin, the lips, the nails, the hair, the eyelashes, the eyebrows and/or the scalp, in particular as care products, anti-sun products and makeup products.

The cosmetic compositions according to the invention may, for example, be used as a makeup product.

The cosmetic compositions according to the invention may, for example, be used as a care product and/or anti-sun product for the face and/or the body, of liquid to semi-liquid consistency, such as milks, more or less rich creams, gel creams, or pastes. They may optionally be packaged in an aerosol and may be in the form of a foam or a spray.

The compositions according to the invention in the form of vaporizable fluid lotions in accordance with the invention are applied to the skin or the hair in the form of fine particles using pressurized devices. The devices in accordance with the invention are well known to those skilled in the art and comprise non-aerosol pump-dispensers or “atomizers”, aerosol containers comprising a propellant and also aerosol pump-dispensers that use compressed air as a propellant. The latter are described in U.S. Pat. Nos. 4,077,441 and 4,850,517.

The compositions packaged as an aerosol in accordance with the invention generally contain conventional propellants such as, for example, hydrofluoro compounds, dichlorodifluoromethane, difluoroethane, dimethyl ether, isobutane, n-butane, propane or trichlorofluoromethane. They are preferably present in amounts ranging from 15% to 50% by weight, relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Solubility of Lipophilic Active Agents, Comparison of Prior Art Solvents and the Solvents of the Invention

### Active Agents Tested

### Active Agent 1: ethyl 4-hydroxyphenylcarbamate

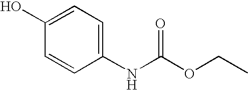

### Active Agent 2: 2-hydroxy-5-octanoylbenzoic acid

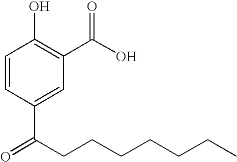

### Active agent 3: N-(2-amino-3-oxidopyrimidin-4-yl)-N′-dodecylurea

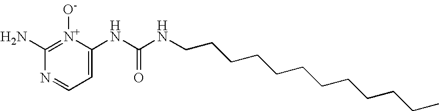

### Screening Agents Tested

### Screen 1: 1-(4-tert-butylphenyl)-3-(2-hydroxyphenyl)propane-1,3-dione

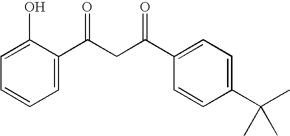

### Screen 2: 1-(4-methoxy-1-benzofuran-5-yl)-3-phenylpropane-1,3-dione

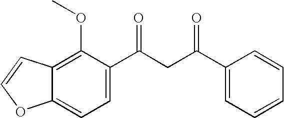

### Screen 3: 1-(4-tert-butylphenyl)-3-(4-methoxyphenyl)propane-1,3-dione (avobenzone) (known under the trademark “Parsol 1789” from DSM):

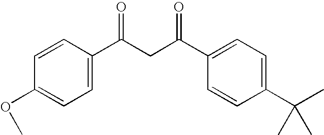

### Screen 4: 1-(4-isopropylphenyl)-3-phenylpropane-1,3-dione

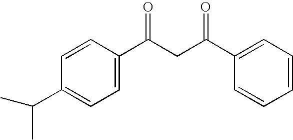

### Screen 5: dineopentyl 2-(3,3-diphenylprop-2-enylidene)malonate

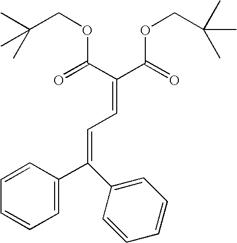

### Screen 6: 2-(2H-1,2,3-benzotriazol-2-yl)-4-methyl-6-(2-methyl-3-{1,3,3,3-tetramethyl-1-[(trimethylsilyl)oxy]disiloxanyl}propyl)phenol

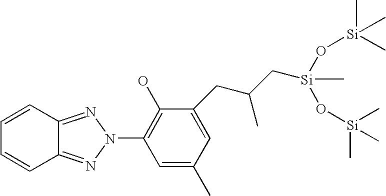

### Screen 7: (2E)-3-(2,4,5-trimethoxyphenyl)prop-2-enoic acid

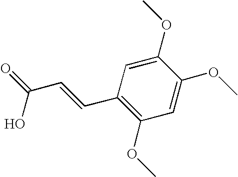

### Screen 8: (2E)-1-(4-hydroxyphenyl)-3-phenylprop-2-en-1-one

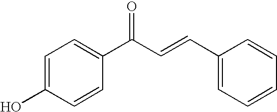

### Comparative Oil 1: Eldew SL-205 from Ajinomoto: Isopropyl N-lauroyl Sarcosinate of Formula

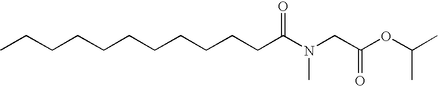

### Comparative Oil 2: Finsolv TN: C₁₂-C₁₅ Alkyl Benzoate

### Comparative Oil 3: Miglyol 812: Caprylic/Capric Acid Triglycerides

### Comparative Oil 4: X-Tend: 2-phenylethyl Benzoate

### Procedure

X mg of product are introduced into Y mg of oil; with mild heating (<60° C.) and the use of a sonicator for 1 minute, the solution obtained is left at laboratory temperature for 1 month; the state of this solution is observed; if no crystal or oil deposit is visible, the solubility of the product is considered to be greater than X×100/(X+Y) weight/weight; when crystals or an oil deposit appear, the test is repeated with 5% less product.

TABLE 1

| Solvent | Active agent 1 | Active agent 2 | Active agent 3 |
|---|---|---|---|
| Isopropyl N- | 23% | 18% | <2% slight |
| lauroyl | | | precipitate |
| sarcosinate | | | |
| Compound (m) | 43% | 45% | 4% |

TABLE 2

| Oil | Screen 1 | Screen 2 | Screen 3 | Screen 4 | Screen 5 | Screen 6 | Screen 7 | Screen 8 |
|---|---|---|---|---|---|---|---|---|
| C₁₂-C₁₅ alkyl | 15% | 25% | 30% | 49% | 28% | 60% | <4% slight | 15% |
| benzoate | | | | | | | precipitate | |
| Compound (m) | 28% | 37% | 44% | 62% | 40% | 80% | 15% | 32% |

TABLE 3

| Solvent | Screen 3 |
|---|---|
| Isopropyl N-lauroyl sarcosinate | 30% |
| C₁₂-C₁₅ alkyl benzoate | 25% |
| Caprylic/capric acid triglycerides | 22% |
| 2-Phenylethyl benzoate | 32% |
| Compound (x) of the invention | 47% |
| Compound (j) of the invention | 50% |
| Compound (l) of the invention | 40% |
| Compound (ii) of the invention | 39% |

### Formulation Examples 1-4

The following formulations were prepared, the amounts are expressed as percentages by weight relative to the total weight of the composition.

| | | | | |
|---|---|---|---|---|
| Composition | Ex 1 | Ex 2 | Ex 3 | Ex 4 |

Phase A

| | | | | |
|---|---|---|---|---|
| Polydimethylsiloxane | 0.5 | 0.5 | 0.5 | 0.5 |
| Preservatives | 1 | 1 | 1 | 1 |
| Stearic acid | 1.5 | 1.5 | 1.5 | 1.5 |
| Glyceryl monostearate/PEG-100 | 1 | 1 | 1 | 1 |
| stearate mixture | | | | |
| Mixture of cetylstearyl glucoside | 2 | 2 | 2 | 2 |
| and of cetyl, stearyl alcohols | | | | |
| Cetyl alcohol | 0.5 | 0.5 | 0.5 | 0.5 |
| Butyl 1-butyl-5-oxopyrrolidine-3- | 20 | | 15 | — |
| carboxylate (compound (m)) | | | | |
| Methyl 1-butyl-5-oxopyrrolidine-3- | | 20 | | 15 |
| carboxylate (compound (j)) | | | | |
| 1-(4-Methoxy-1-benzofuran-5-yl)- | 5 | 5 | | |
| 3-phenylpropane-1,3-dione | | | | |
| Dineopentyl 2-(3,3-diphenylprop- | — | — | 5 | 5 |
| 2-enylidene)malonate | | | | |

Phase B

| | | | | |
|---|---|---|---|---|
| Deionized water | qs 100 | qs 100 | qs 100 | qs 100 |
| Glycerol | 5 | 5 | 5 | 5 |
| Xanthan gum | 0.2 | 0.2 | 0.2 | 0.2 |
| Monocetyl phosphate | 1 | 1 | 1 | 1 |

Phase C

| | | | | |
|---|---|---|---|---|
| Isohexadecane | 1 | 1 | 1 | 1 |
| Acrylic acid/stearyl methacrylate | 0.2 | 0.2 | 0.2 | 0.2 |
| copolymer | | | | |
| Triethanolamine | qs pH | qs pH | qs pH | qs pH |

Procedure:

The aqueous phase (phase B) containing all its ingredients is heated to 80° C. in a water bath. The fatty phase (phase A) containing all its ingredients is heated to 80° C. in a water bath. A is emulsified in B with rotor-stator stirring (instrument from the company Moritz). Phase C is incorporated and the resulting mixture is left to return to ambient temperature with moderate stirring. The triethanolamine is introduced so as to adjust the pH to the desired value at the end of production. The anti-sun emulsions obtained are stable with respect to storage and do not display any crystals or precipitates.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable UV-screening cosmetic composition comprising at least one ester solvent derived from 4-carboxy-2-pyrrolidinone, having the formula (I): 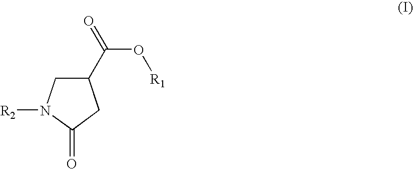
in which:
R₁ is a linear or branched C₁-C₂₀ alkyl radical, and
R₂ is a linear or branched C₁-C₂₀ alkyl radical optionally containing a C₅-C₆ ring member, the phenyl radical, the benzyl radical or the phenethyl radical, and at least one liquid fatty phase and at least one lipophilic UV-screening active agent, said at least one ester derivative comprising a solvent for said at least one active agent in said at least one liquid fatty phase and/or comprising an agent for enhancing the solubility of said at least one active agent in said fatty phase, formulated into a topically applicable, cosmetically acceptable medium therefor.

2. A cosmetic composition as defined by claim 1, in which the compounds of formula (I) are selected from among the following compounds (a) to (oo):

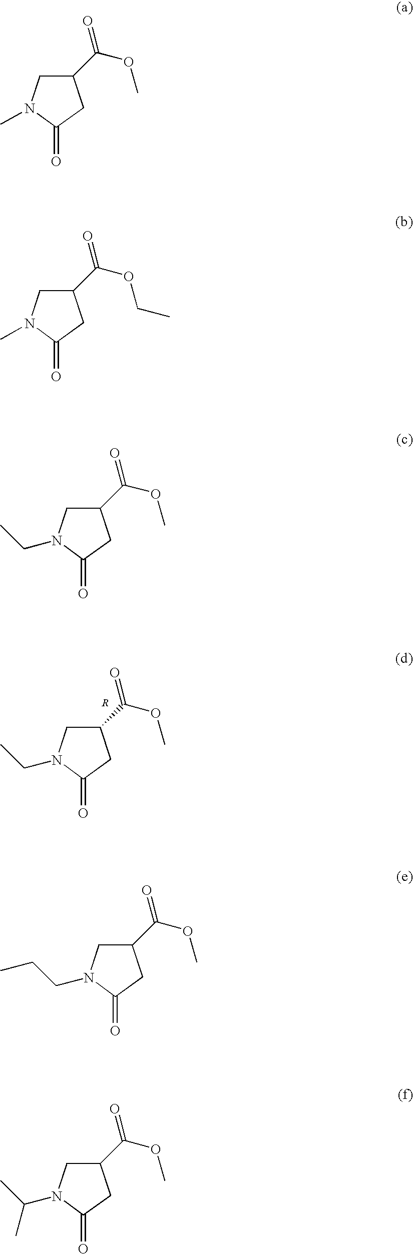

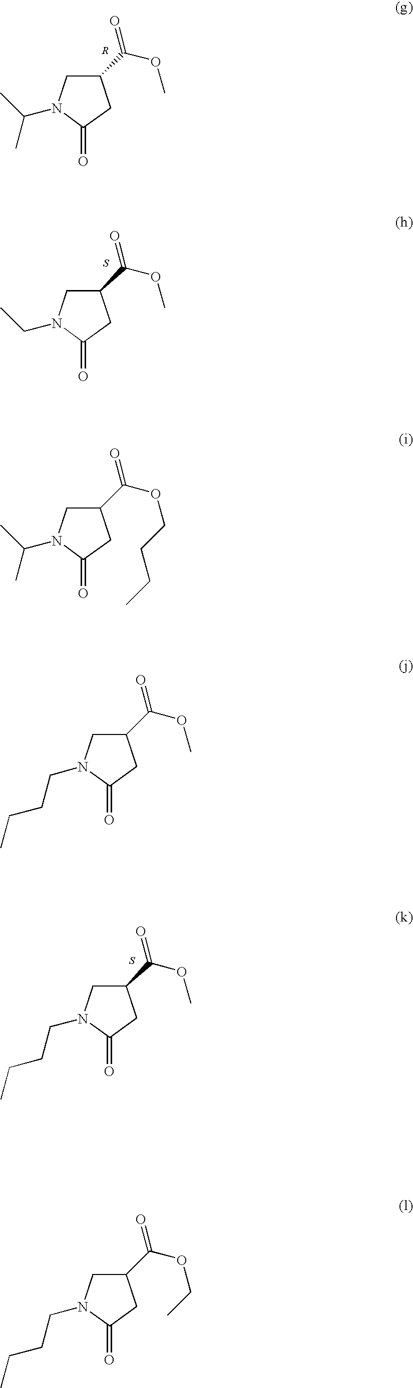

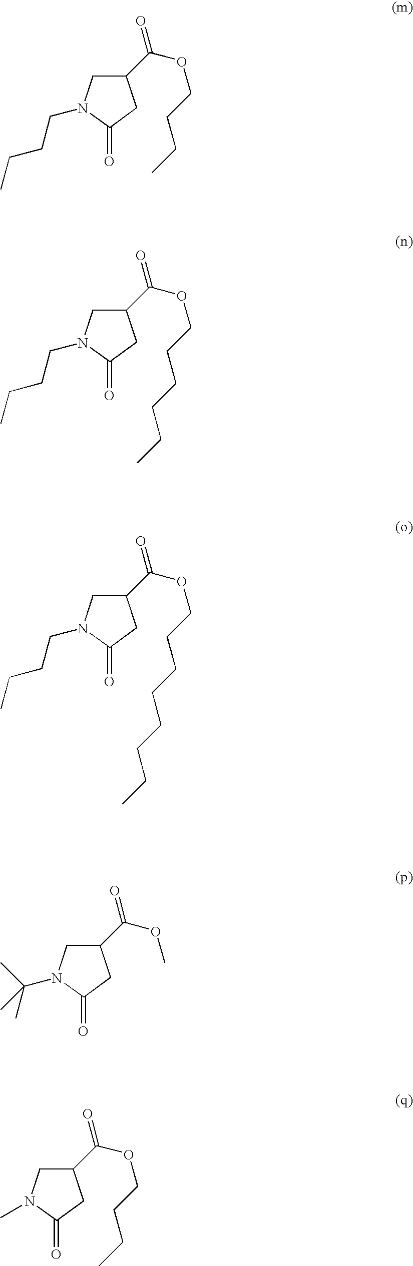

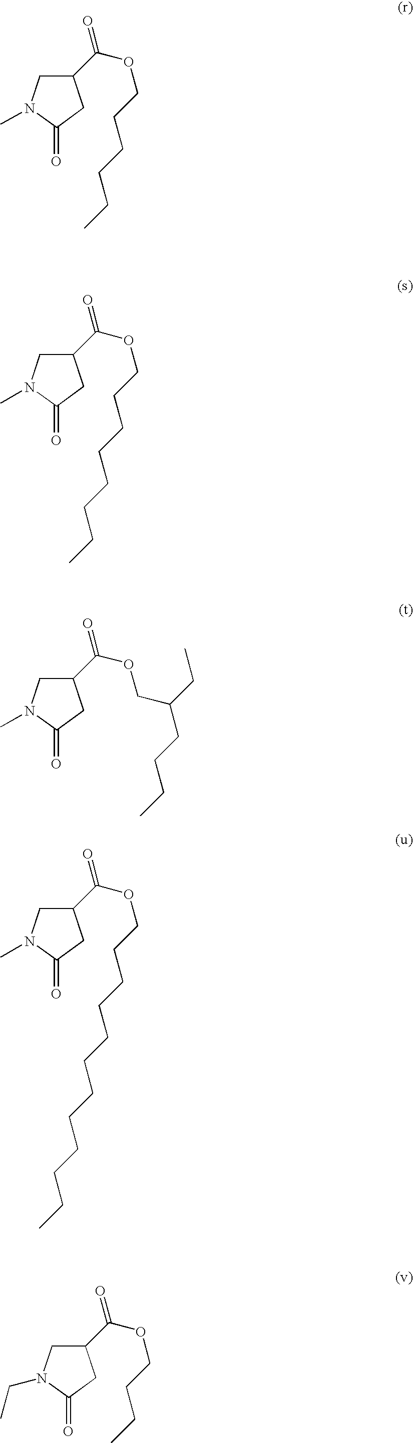

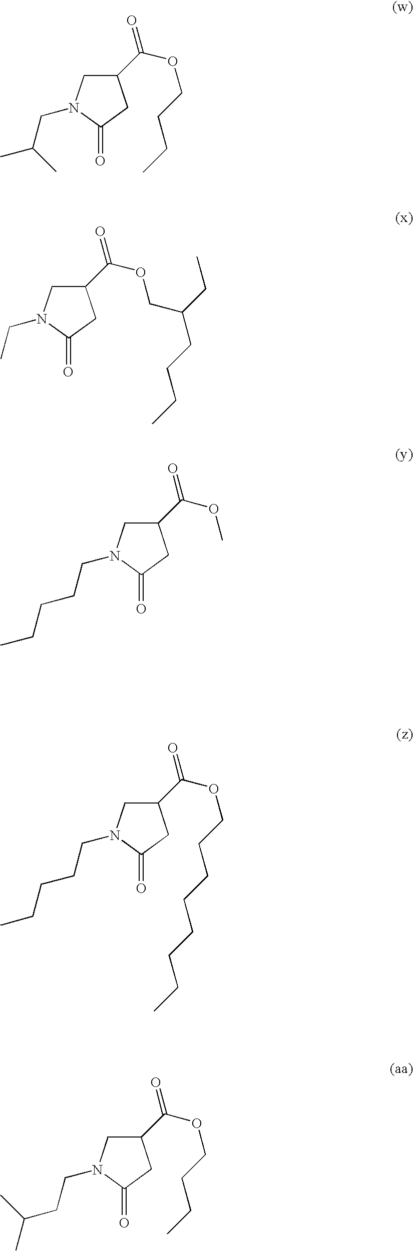

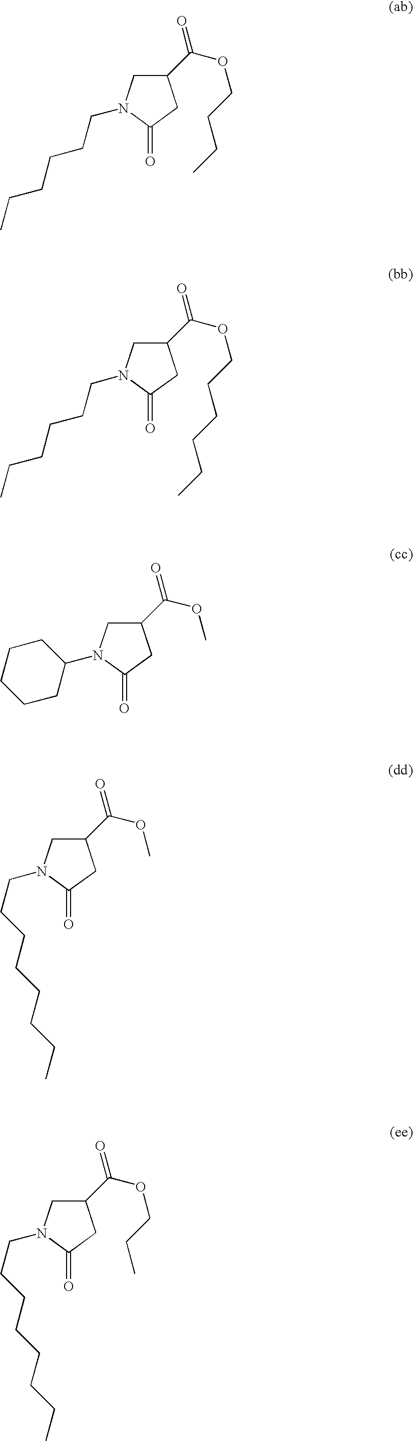

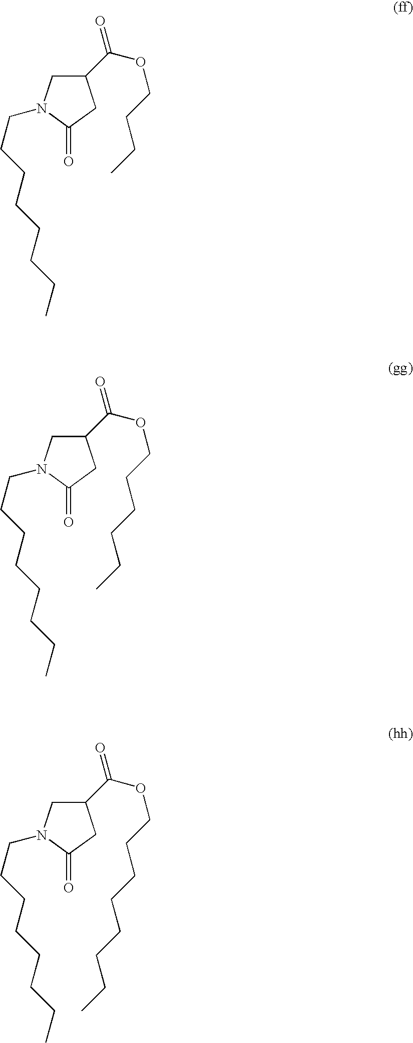

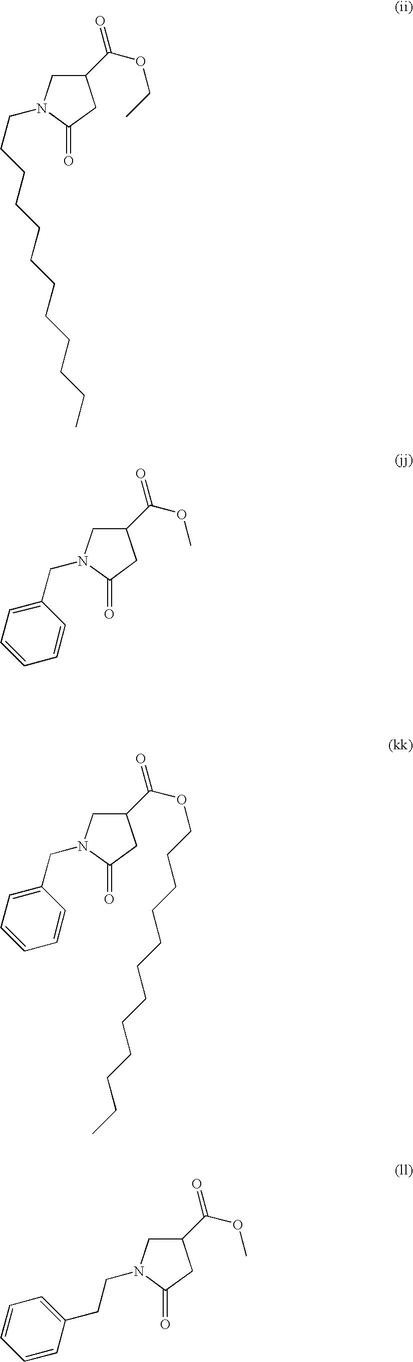

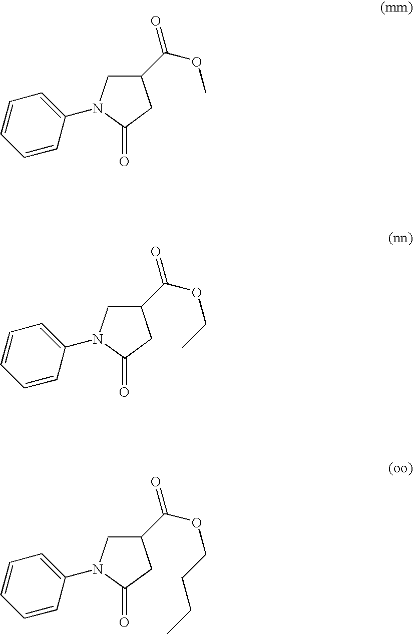

3. A cosmetic composition as defined by claim 2, in which the compounds of formula (I) are selected from among the compounds (t), (u), (v), (x), (j), (l), (m), (w), (n), (ab), (ii) and (kk).

4. A cosmetic composition as defined by claim 1, in which the derivative(s) of formula (I) constitute(s) the sole solvent for the lipophilic active agent(s).

5. A cosmetic composition as defined by claim 1, in which the at least one lipophilic active agent is selected from among lipophilic organic UV screening agents.

6. A cosmetic composition as defined by claim 5, in which the at least one lipophilic screening agent is selected from among para-aminobenzoic acid derivatives, salicylic derivatives, cinnamic derivatives, anthranilic derivatives, dibenzoylmethane derivatives, β,β-diphenylacrylate derivatives, benzylidenecamphor derivatives, phenylbenzimidazole derivatives, benzotriazole derivatives, imidazoline derivatives, benzalmalonate derivatives, 4,4-diarylbutadiene derivatives, benzoxazole derivatives, chalcones, malonitrile or malonate diphenyl butadiene derivatives, and mixtures thereof.

7. A cosmetic composition as defined by claim 6, in which the lipophilic organic UV screening agents are selected from among dibenzoylmethane derivatives, benzotriazoles, chalcones, and malonitrile or malonate diphenyl butadiene derivatives.

8. A cosmetic composition as defined by claim 1, in which the at least one lipophilic active agent is selected from among aminophenol derivatives, salicylic acid derivatives, N,N′-di(arylmethylene)ethylenediaminetriacetate derivatives, 2-amino-4-alkylaminopyrimidine 3-oxide derivatives, flavonoids, retinol and its derivatives, carotenoids, lycopene, and also fragrances, essential oils, hormones, vitamins, vitamin E, ceramides, or mixtures thereof.

9. A cosmetic composition as defined by claim 1, in which the derivative(s) of formula (I) is (are) present therein at contents ranging from 1% to 30% by weight, relative to the total weight of the composition.

10. A method for enhancing the effectiveness of at least one lipophilic UV-screening active agent and/or for enhancing the cosmetic qualities and/or the stability of cosmetic compositions comprised thereof, which comprises formulating same with at least one ester solvent derived from a 4-carboxy-2-pyrrolidinone as defined by claim 1.

11. A method for improving the sun protection factor of at least one lipophilic UV-screening active agent, comprising formulating same with at least one ester solvent derived from a 4-carboxy-2-pyrrolidinone as defined by claim 1.

12. A regime or regimen for photoprotecting the skin against the damaging effects of UV-radiation, comprising topically applying thereon a thus effective amount of the stable UV-screening cosmetic composition as defined by claim 1.
